(19) United States
(12) Patent Application Publication
(10) Pub. No.: US 2011/0155242 A1
(43) Pub. Date: Jun. 30, 2011

(54) FLUORESCENT MATERIALS AND SOLAR CELLS THEREWITH

(75) Inventors: Ching-Yen Wei, Taipei (TW); Yi-Ling Chen, Sanchong City (TW); Wei-Jen Liu, Taoyuan City (TW); Yi-Chen Chiu, Hsinchu (TW); Yang-Fang Chen, Taipei (TW); Kao-Chiang Hsu, Pingzhen City (TW); Yu-Wei Tai, Hsinchu (TW); Chia-Ching Wang, Zhubei City (TW); Meng-Hsiu Wu, Yonghe City (TW)

(73) Assignees: INDUSTRIAL TECHNOLOGY RESEARCH INSTITUTE, Hsinchu County (TW); NEO SOLAR POWER CORP, Hsinchu (TW)

(21) Appl. No.: 12/753,874

(22) Filed: Apr. 3, 2010

(30) Foreign Application Priority Data
Dec. 30, 2009 (TW) 098145764

(51) Int. Cl. H01L 31/00; C07D 333/52; C07D 311/74

(52) U.S. Cl. 136/257; 549/58; 549/400

(57) ABSTRACT

A fluorescent material of Formula (I) is provided.

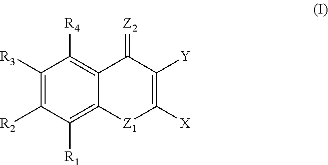

In Formula (I), all the variables thereof are described in the specification. The invention also provides a solar cell with the disclosed fluorescent material. The solar cell with the fluorescent material includes a solar cell and a fluorescent layer including the disclosed fluorescent material of Formula (I) coating on the solar cell.

### CROSS REFERENCE TO RELATED APPLICATIONS

This Application claims priority of Taiwan Patent Application No. 98145764, filed on Dec. 30, 2009, the entirety of which is incorporated by reference herein.

### BACKGROUND OF THE INVENTION

1. Field of the Invention

The invention relates to a fluorescent material, and more particularly to a fluorescent material capable of absorbing ultraviolet light and emitting visible light (yellow light).

2. Description of the Related Art

Fundamentally, photovoltaic cells can only convert part of the incident sunlight into electrical energy; a large portion of the energy is lost in the form of heat. For example, a silicon solar cell can absorb all photons which have an energy above the band edge of 1.1 eV of crystalline silicon, i.e. a wavelength of .ltoreq.1300 nm. The excess energy of the absorbed photons is converted to heat and leads to heating of the photovoltaic cell. This reduces its efficiency.

Therefore, as early as the 1970s, fluorescence conversion cells were described which are a combination of photovoltaic cells with fluorescent light collecting systems (solar collectors) and enable better utilization of energy from sunlight. The solar collectors convert the absorbed sunlight to light which is of a longer wavelength but is still above the silicon band edge in energetic terms and thus reduce the heating of the photovoltaic cell. The use of a plurality of fluorescers which absorb and emit at different wavelengths (known as cascades) allows the incident sunlight to be converted particularly effectively to light energy which is suitable for the photovoltaic cell.

Several fluorescence conversion solar cells are disclosed, for example U.S. Pat. No. 4,367,367 describes fluorescence conversion solar cells based on a plurality of glass plates doped with fluorescent metal ions such as UO₂²⁺, Eu³⁺, Cr³⁺, Yb³⁺ and Nd³⁺, and coated with fluorescent dyes (violanthrone, Rhodamine 6G) in PMMA matrix.

### BRIEF SUMMARY OF THE INVENTION

One embodiment of the invention provides a fluorescent material of Formula (I):

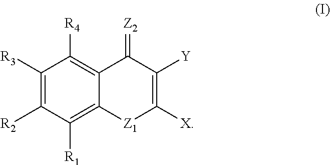

In Formula (I), R₁ to R₄ are, independently, hydrogen, fluorine, chlorine, bromine, cyano, hydroxyl, C1-C12 alkyl or C1-C12 alkoxyl, Z₁ and Z₂ are oxygen, sulfur or selenium, Y is hydroxyl or hydrosulfide group, and X is

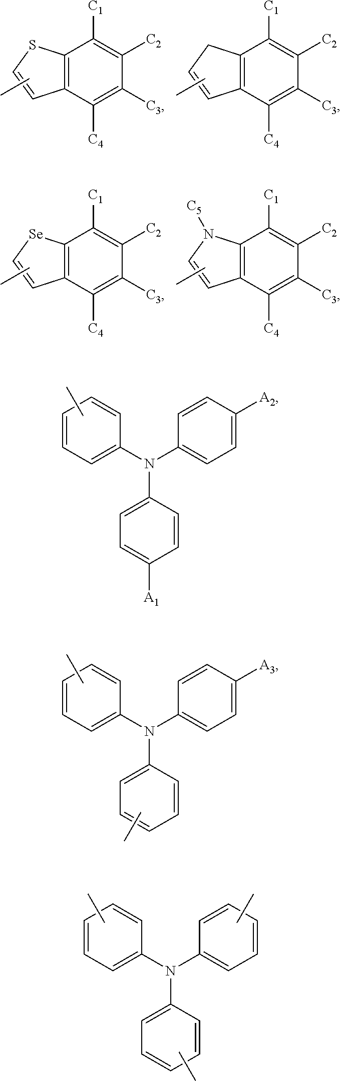

or —N(CₙH₂ₙ₊₁)₂ (n=0-6), wherein C₁ to C₄ and A₁ to A₃ are, independently, hydrogen, fluorine, chlorine, bromine, cyano, hydroxyl, C1-C12 alkyl or C1-C12 alkoxyl, and C₅ is hydrogen or C1-C12 alkyl.

One embodiment of the invention provides a solar cell with a fluorescent material comprising a solar cell and a fluorescent layer comprising the disclosed fluorescent material of Formula (I) coating on the solar cell.

The invention provides the fluorescent layer blended with the modified fluorescent material coated on solar cells to improve cell efficiency. The fluorescent material with excited state intramolecular proton transfer characteristics is isomerized and emits long-wavelength fluorescent light (yellow light) within the visible light region after absorbing ultraviolet light with a wavelength of 350 to 400 nm. The energy of the visible light emitted from the fluorescent material through the excited state intramolecular proton transfer and the absorption band of the light transfer layer are further mutually overlapped to generate resonance energy transfer. Thus, the disclosed fluorescent material blended in the fluorescent layer improves light energy retransfer, cell efficiency.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a schematic diagram showing a solar cell structure according to an embodiment of the invention;

FIG. 2 is an absorption and fluorescence spectrum of the fluorescent material (I-1) of the invention;

FIG. 3 is an absorption and fluorescence spectrum of the fluorescent material (I-2) of the invention; and

FIG. 4 shows absorption ranges of light transfer layers of various solar cells.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides a fluorescent material of Formula (I):

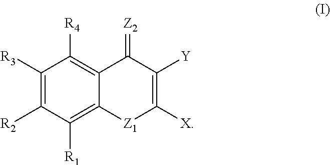

In Formula (I), R₁ to R₄ may be, independently, hydrogen, fluorine, chlorine, bromine, cyano, hydroxyl, C1-C12 alkyl or C1-C12 alkoxyl, preferably C4-8 alkyl or C4-8 alkoxyl. Z₁ and Z₂ may be oxygen, sulfur or selenium. Y may be hydroxyl or hydrosulfide group. X may be

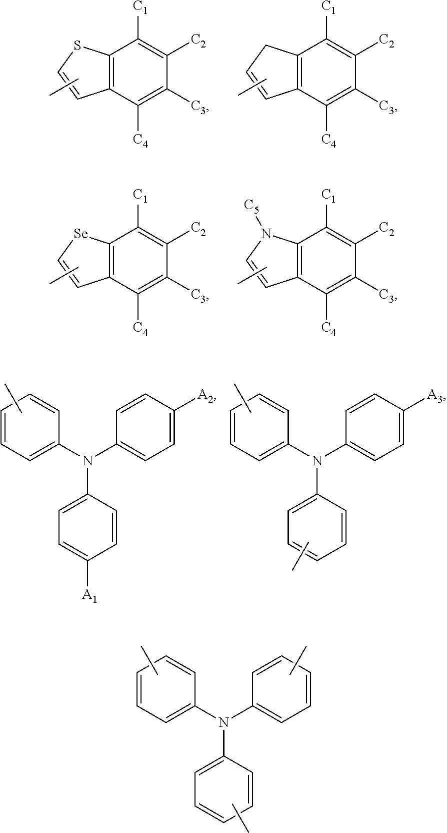

or —N(CₙH₂ₙ₊₁)₂ (n=0-6). C₁ to C₄ and A₁ to A₃ may be, independently, hydrogen, fluorine, chlorine, bromine, cyano, hydroxyl, C1-C12 alkyl or C1-C12 alkoxyl, preferably C4-8 alkyl or C4-8 alkoxyl. C₅ may be hydrogen or C1-C12 alkyl, preferably C4-8 alkyl.

The fluorescent material has an absorption wavelength of about 350 nm to 400 nm.

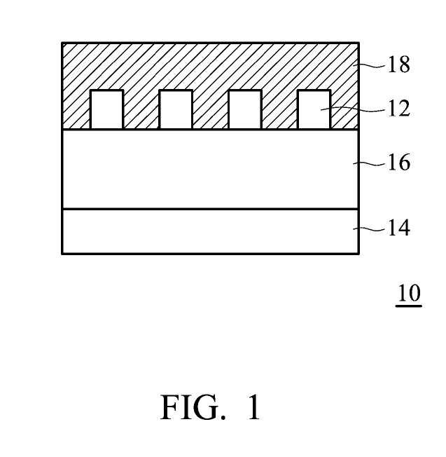

Referring to FIG. 1, according to an embodiment of the invention, a solar cell with a fluorescent material is provided. The solar cell 10 comprises an upper electrode 12, a lower electrode 14, a light transfer layer 16 and a fluorescent layer 18. The upper electrode 12 is opposed to the lower electrode 14. The light transfer layer 16 is disposed between the upper electrode 12 and the lower electrode 14. The upper electrode 12 may be a patterned transparent electrode, for example indium tin oxide (ITO) or fluorine tin oxide (SnO₂:F, FTO) or a metal electrode, for example silver or aluminum. The lower electrode 14 may be a metal electrode, for example silver or aluminum. The light transfer layer 16 may comprise crystalline silicon, amorphous silicon, gallium arsenide (GaAs), cadmium telluride (CdTe) or copper indium selenide (CIS). The fluorescent layer 18 comprises the disclosed fluorescent material of Formula (I). The fluorescent layer 18 may be coated on the upper electrode 12 and filled therebetween.

The fluorescent layer 18 may further comprises BM12 (35 to 45 wt % polyester dissolved in carbitol acetate), poly(ethyl methacrylate) (PMMA), ethylene vinyl alcohol (EVA) or poly(vinyl butyral) (PVB).

The invention provides the fluorescent layer blended with the modified fluorescent material coated on solar cells to improve cell efficiency. The fluorescent material with excited state intramolecular proton transfer characteristics is isomerized and emits long-wavelength fluorescent light (yellow light) within the visible light region after absorbing ultraviolet light with a wavelength of 350 to 400 nm. The energy of the visible light emitted from the fluorescent material through the excited state intramolecular proton transfer and the absorption band of the light transfer layer are further mutually overlapped to generate resonance energy transfer. Thus, the disclosed fluorescent material blended in the fluorescent layer improves light energy retransfer, and cell efficiency.

### Example 1

### Synthesis of the Fluorescent Material (I-1) of the Invention

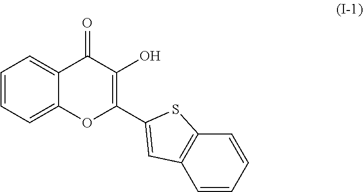

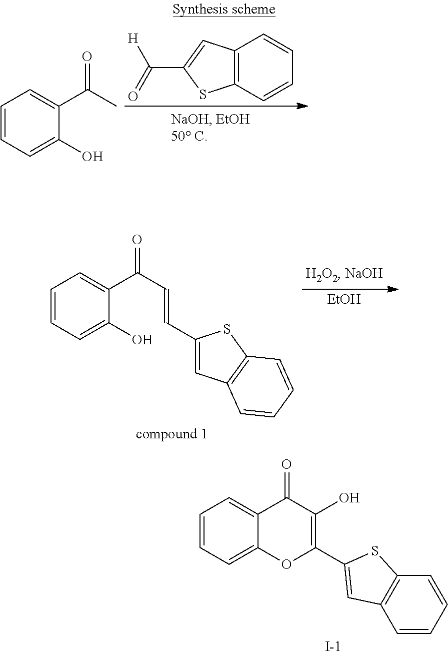

2.0 ml of 2′-Hydroxyacetophenone (15 mmol) was added to 2 g of sodium hydroxide aqueous solution (10 ml of water) and 50 ml of ethanol. After being completely dissolved, a canary-yellow clear solution was prepared. Next, 15 mmol of benzo[b]thiophene-2-carbaldehyde was added to the clear solution and stirred for 8 hours at room temperature. Compound 1 solution was formed. 5 ml of hydrogen peroxide (30%) was then added to compound 1 solution and stirred for 12 hours at room temperature. Proper hydrochloric acid aqueous solution was then added to compound 1 solution to neutralize the solution. Compound 1 solution was then extracted by adding dichloromethane and purified by column using dichloromethane as an eluent. The fluorescent material I-1 was finally obtained.

¹H NMR (200 MHz, CDCl₃): 7.39-7.44 (3H, m), 7.60-7.62 (1H, m), 7.70-7.72 (1H, m), 7.89-7.91 (2H, m), 8.23-8.25 (1H, m), 8.28 (1H, s).

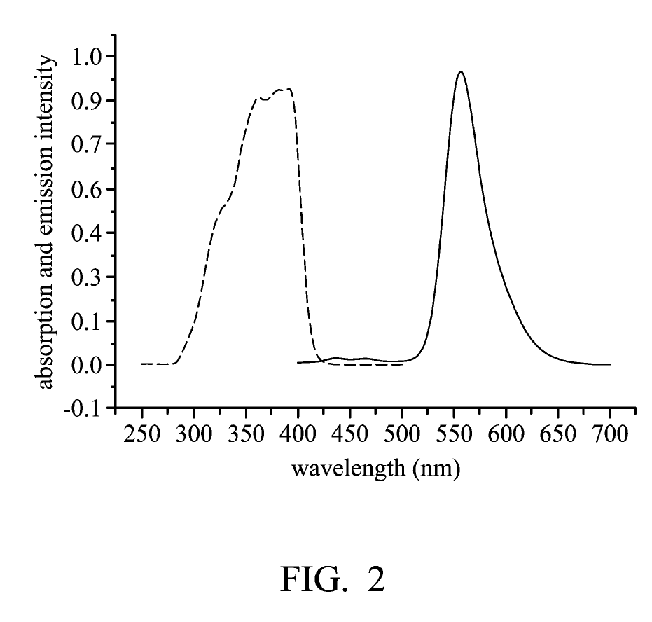

FIG. 2 is an absorption and fluorescence spectrum of the fluorescent material (I-1). The figure indicates that the fluorescent material (I-1) absorbed the light with wavelengths of 350 nm to 400 nm and emitted the fluorescent light with wavelengths of 560 nm to 600 nm.

### Example 2

### Synthesis of the Fluorescent Material (I-2) of the Invention

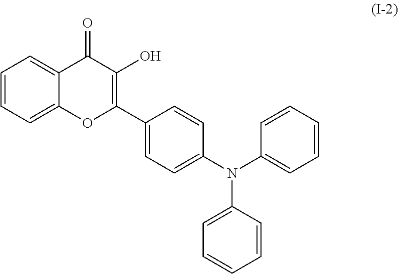

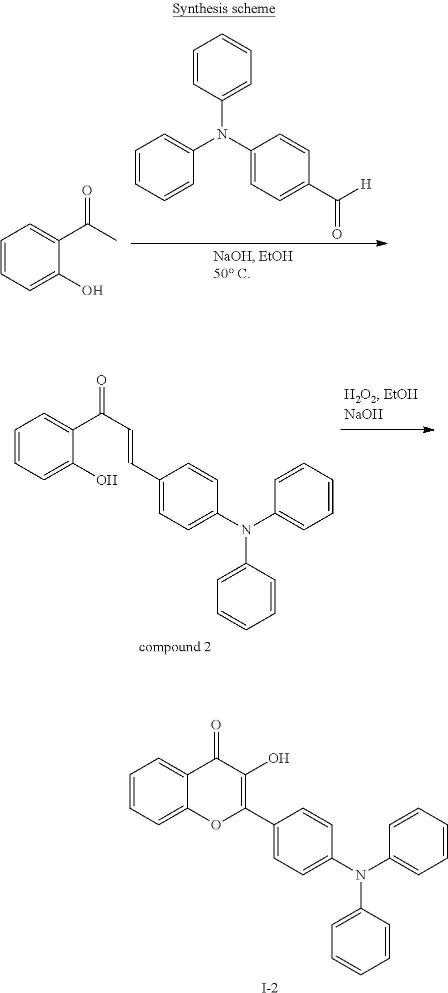

2.0 ml of 2′-Hydroxyacetophenone (15 mmol) was added to 2 g of sodium hydroxide aqueous solution (10 ml of water) and 50 ml of ethanol. After completely dissolving, a canary-yellow clear solution was prepared. Next, 15 mmol of 4-Diphenylamino-benzaldehyde was added to the clear solution and stirred for 8 hours at room temperature. Compound 2 solution was formed. 5 ml of hydrogen peroxide (30%) was then added to compound 2 solution and stirred for 12 hours at room temperature. Proper hydrochloric acid aqueous solution was then added to compound 2 solution to neutralize the solution. Compound 2 solution was then extracted by adding dichloromethane and purified by column using dichloromethane as an eluent. The fluorescent material I-2 was finally obtained.

¹H NMR (200 MHz, CDCl₃): 7.02-7.16 (9H, m), 7.27-7.31 (4H, m), 7.38 (1H, t), 7.52 (1H, d), 7.63-7.67 (1H, m) 8.09-8.11 (1H, m).

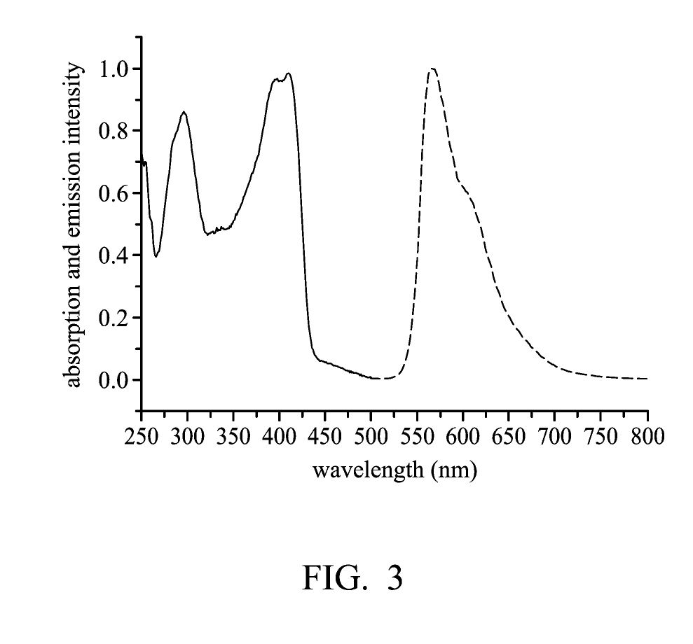

FIG. 3 is an absorption and fluorescence spectrum of the fluorescent material (I-2). The figure indicates that the fluorescent material (I-2) absorbed the light with wavelengths of 350 nm to 400 nm and emitted the fluorescent light with wavelengths of 560 nm to 600 nm.

### Example 3

### Preparation of the Solar Cell I of the Invention

A silicon solar cell prepared from a silicon substrate was provided. Next, 10 g of BM12 (35 to 45 wt % polyester dissolved in carbitol acetate) (purchased from Exojet Technology Corporation, type: BM12) and 0.12 g of the fluorescent material I-1 (prepared from Example 1) were blended and uniformly stirred with a magnetite for 2 hours to form a slurry. The slurry was then coated on the silicon solar cell by screen printing. After baking at 70° C. for 3 hours, the solar cell I was prepared. Next, the efficiency of the solar cell was tested.

### Example 4

### Preparation of the Solar Cell II of the Invention

A silicon solar cell prepared from a silicon substrate was provided. Next, 10 g of BM12 (35 to 45 wt % polyester dissolved in carbitol acetate) (purchased from Exojet Technology Corporation, type: BM12) and 0.04 g of the fluorescent material I-2 (prepared from Example 2) were blended and uniformly stirred with a magnetite for 2 hours to form a slurry. The slurry was then coated on the silicon solar cell by screen printing. After baking at 70° C. for 3 hours, the solar cell II was prepared. Next, the efficiency of the solar cell was tested.

### Comparative Example 1

### Preparation of a Conventional Solar Cell

A silicon solar cell prepared from a silicon substrate was provided. Next, 10 g of BM12 (35 to 45 wt % polyester dissolved in carbitol acetate) (purchased from Exojet Technology Corporation, type: BM12) was uniformly stirred with a magnetite for 2 hours to form a slurry. The slurry was then coated on the silicon solar cell by screen printing. After baking at 70° C. for 3 hours, the solar cell was prepared. Next, the efficiency of the solar cell was tested.

### Example 5

### Comparison of Cell Efficiency Between Various Solar Cells

The cell efficiency of the disclosed solar cells coated with a fluorescent layer blended with the fluorescent material I-1 and I-2 and a conventional solar cell without coating with fluorescent layer was compared in Table 1.

TABLE 1
Coating with BM12

| States | Cell efficiency |
|---|---|
| Before coating | 15.20% |
| After coating | 15.11% |
| Deterioration | −0.09% |

Table 1 shows cell efficiency of a 6-inch silicon solar cell coated with commercial slurry BM12.

Table 1 indicates that the cell efficiency of the silicon solar cell was reduced 0.09% after coating.

TABLE 2
Coating with fluorescent layer

| States | Cell efficiency |
|---|---|
| Before coating | 16.25% |
| After coating | 16.39% |
| Improvement | +0.14% |

Table 2 shows cell efficiency of a 6-inch silicon solar cell coated with a fluorescent layer (blended with commercial slurry BM12 and 0.4 wt % fluorescent material I-1).

Table 2 indicates that the cell efficiency of the silicon solar cell was improved 0.14% after coating.

TABLE 3
Coating with BM12

| States | Cell efficiency |
|---|---|
| Before coating | 16.49% |
| After coating | 16.44% |
| Deterioration | −0.05% |

Table 3 shows cell efficiency of a 6-inch silicon solar cell coated with commercial slurry BM12.

Table 3 indicates that the cell efficiency of the silicon solar cell was reduced 0.05% after coating.

TABLE 4
Coating with fluorescent layer

| States | Cell efficiency |
|---|---|
| Before coating | 16.42% |
| After coating | 16.48% |
| Improvement | +0.06% |

Table 4 shows cell efficiency of a 6-inch silicon solar cell coated with a fluorescent layer (blended with commercial slurry BM12 and 0.4 wt % fluorescent material I-2).

Table 4 indicates that the cell efficiency of the silicon solar cell was improved 0.06% after coating.

From Tables 1 to 4, the disclosed solar cells coated with the fluorescent layer blended with the fluorescent material I-1 or I-2 absorbed a great quantity of ultraviolet light source and emitted visible light. The absorption of visible light of the light transfer layer was thus increased such that the light source was effectively utilized via reabsorption from the light transfer layer of the solar cells. Thus, the cell efficiency of the disclosed solar cells was apparently superior to that of a conventional solar cell without the fluorescent coating material.

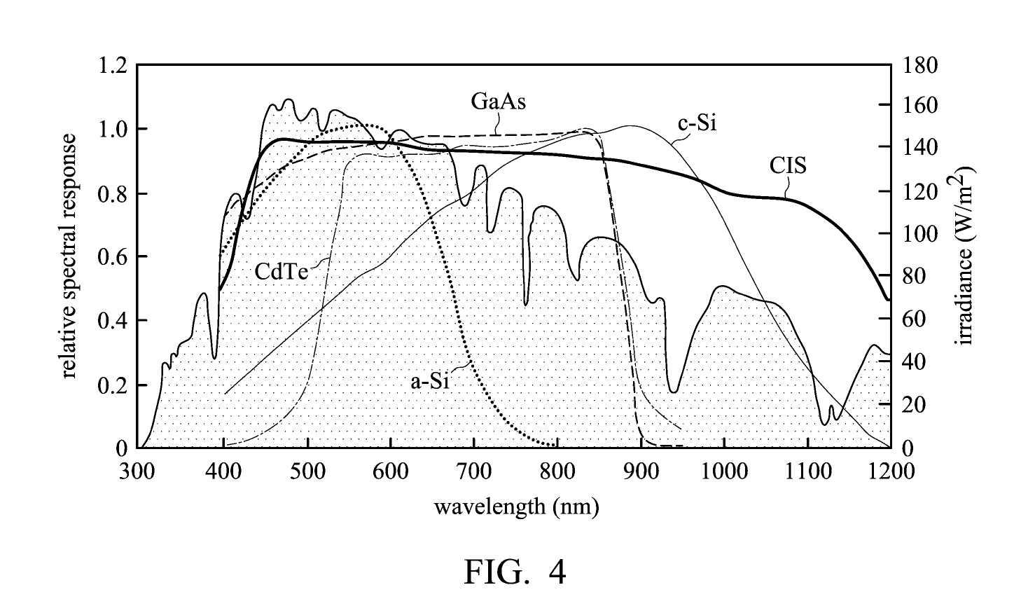

Additionally, referring to FIG. 4, the absorption ranges of light transfer layers of various solar cells were shown. The figures indicates that, in addition to crystalline silicon (Examples 3 and 4), the disclosed fluorescent layer was also suitable for coating on other light transfer layers, for example amorphous silicon, gallium arsenide (GaAs), cadmium telluride (CdTe) or copper indium selenide (CIS), thereby increasing absorption energy of such light transfer layers.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A fluorescent material of Formula (I): 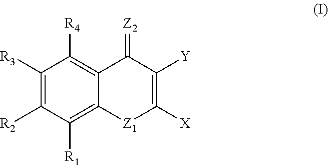
wherein
R₁ to R₄ are, independently, hydrogen, fluorine, chlorine, bromine, cyano, hydroxyl, C1-C12 alkyl or C1-C12 alkoxyl;
Z₁ and Z₂ are oxygen, sulfur or selenium;
Y is hydroxyl or hydrosulfide group; and
X is 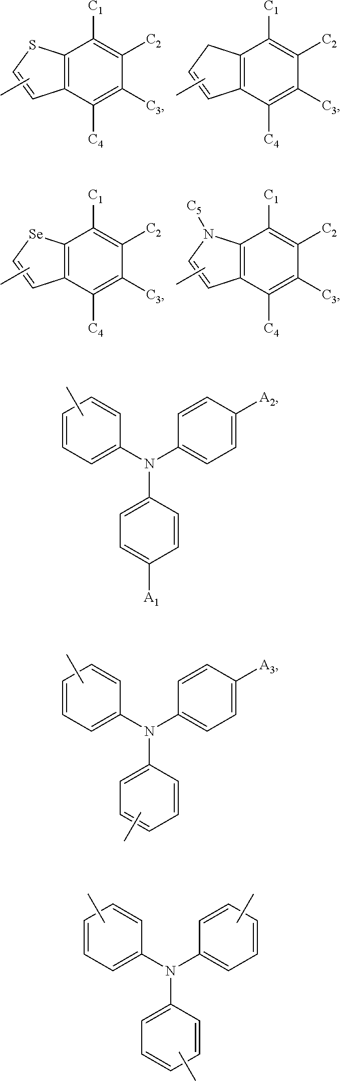
or —N(CₙH₂ₙ₊₁)₂ (n=0-6), wherein C₁ to C₄ and A₁ to A₃ are, independently, hydrogen, fluorine, chlorine, bromine, cyano, hydroxyl, C1-C12 alkyl or C1-C12 alkoxyl, and C₅ is hydrogen or C1-12 alkyl.

2. The fluorescent material as claimed in claim 1, wherein R₁ to R₄ are, independently, C4-8 alkyl or C4-8 alkoxyl.

3. The fluorescent material as claimed in claim 1, wherein C₁ to C₄ and A₁ to A₃ are, independently, C4-8 alkyl or C4-8 alkoxyl.

4. The fluorescent material as claimed in claim 1, wherein C₅ is C4-8 alkyl.

5. A solar cell with a fluorescent material, comprising:
a solar cell; and
a fluorescent layer comprising a fluorescent material as claimed in claim 1 coating on the solar cell.

6. The solar cell with a fluorescent material as claimed in claim 5, wherein the solar cell comprises an upper electrode, a lower electrode opposed to the upper electrode and a light transfer layer disposed between the upper electrode and the lower electrode.

7. The solar cell with a fluorescent material as claimed in claim 6, wherein the upper electrode is a transparent electrode.

8. The solar cell with a fluorescent material as claimed in claim 7, wherein the upper electrode is a patterned electrode.

9. The solar cell with a fluorescent material as claimed in claim 8, wherein the fluorescent layer is coated on the upper electrode and filled therebetween.

10. The solar cell with a fluorescent material as claimed in claim 6, wherein the lower electrode is a metal electrode.

11. The solar cell with a fluorescent material as claimed in claim 6, wherein the light transfer layer comprises crystalline silicon, amorphous silicon, gallium arsenide (GaAs), cadmium telluride (CdTe) or copper indium selenide (CIS).

12. The solar cell with a fluorescent material as claimed in claim 5, wherein the fluorescent layer further comprises poly(ethyl methacrylate) (PMMA), ethylene vinyl alcohol (EVA) or poly(vinyl butyral) (PVB).
